(19) United States
(12) Patent Application Publication
(10) Pub. No.: US 2009/0246671 A1
(43) Pub. Date: Oct. 1, 2009

(54) DEVELOPER AND PROCESSING METHOD FOR LIGHT SENSITIVE PLANOGRAPHIC PRINTING PLATE MATERIAL

(75) Inventor: Toshitsugu Suzuki, Tokyo (JP)

Correspondence Address: CANTOR COLBURN, LLP, 20 Church Street, 22nd Floor, Hartford, CT, 06103, US

(73) Assignee: KONICA MINOLTA MEDICAL & GRAPHIC, INC., Tokyo, (JP)

(21) Appl. No.: 12/064,888

(22) PCT Filed: Aug. 11, 2006
(86) PCT No.: PCT/JP2006/315906

§ 371 Date: Feb. 26, 2008

(30) Foreign Application Priority Data
Aug. 30, 2005 (JP) JP2005-249024

(51) Int. Cl. G03G 9/08; C07C 229/24; G03G 9/00; G03F 7/20

(52) U.S. Cl. 430/108.11; 560/170; 560/171; 430/108.2; 430/302

(57) ABSTRACT

The invention provides a developer and a processing method for a light sensitive planographic printing plate material (also referred to simply as a planographic printing plate material) providing excellent contamination resistance during printing and high reproduction of dot and line images. The developer for a light sensitive planographic printing plate material comprises an aluminum support and provided thereon, a light sensitive layer comprised of a light sensitive resin composition, the light sensitive planographic printing plate material after exposure being developed with the developer, and is characterized in that it contains a compound represented by the following formula (1),

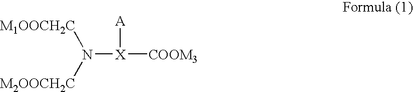

### FIELD OF THE INVENTION

The present invention relates to a developer and a processing method for a light sensitive printing plate material, and particularly to a developer and a processing method for a light sensitive printing plate material providing excellent contamination resistance during printing and high reproduction of dot and line images.

### TECHNICAL BACKGROUND

Lithography is a printing method employing the characteristics in which oil is immiscible with water. On a planographic printing plate are formed image portions, which repel water and receive oleophilic ink, and non-image portions, which repel oleophilic ink and receive water. Then, lithographic printing is carried out in which only oleophilic ink received on the image portions of such a planographic printing plate is transferred to a paper sheet. As a printing press for lithographic printing is generally used an off-set printing press in which the oleophilic ink received on the image portions is once transferred onto a blanket, and then the ink on the blanket is further transferred onto a paper sheet.

A light sensitive planographic printing plate material comprises a support and provided thereon, a light sensitive layer. As the support of the light sensitive planographic printing plate material, one having high hydrophilicity, high water retention property, and excellent adhesion to the light sensitive layer is desired, and an aluminum plate whose surface is subjected to roughening treatment is generally used.

As a method for surface roughening a support is known a mechanical surface-roughening method such as a ball graining method, a brush graining method, a blast graining method, a buffing method or a horning method, or a electro-chemical method in which the support surface is electrically roughened in an electrolyte solution of an acid such as hydrochloric acid or nitric acid through a direct current or an alternating current. The surface of an aluminum plate treated according to the method described above is relatively soft and is likely to be abraded, the surface-roughened aluminum plate is subjected to anodization treatment to form an anodization film on the surface. The resulting aluminum plate surface is hard and excellent in abrasion resistance.

An exposed planographic printing plate material is developed with an alkaline aqueous solution to remove a light sensitive layer at exposed or unexposed portions and unveil the support surface. The support surface is generally hydrophilic, and the support surface unveiled by development (corresponding to non-image portions) receives water and repels oil-based ink. In contrast, portions (corresponding to image portions), in which the light sensitive layer has not been removed by development, are generally oleophilic, and receive oil-based ink and repel water.

In a printing field in which a relatively high printing durability is required, a planographic printing plate material is used which comprises an aluminum support and provided thereon, a light sensitive layer containing a photopolymerizable light sensitive layer. As the developer for the planographic printing plate material comprising a light sensitive layer containing a photopolymerizable light sensitive layer, an alkali solution is generally used.

As the alkali developer, a solution containing alkali metal silicates is known (see Patent documents 1 and 2 below). When the solution containing alkali metal silicates has high pH, it has problem in that the developer is likely to damage image portions, while when it has a low pH, it has problems in that the developer is likely to gel and produce precipitates.

As a developer containing no silicates, there is known a developer containing an alkali agent, a complexing agent, an anionic surfactant, an emulsifying agent, or an n-alkanoic acid (see for example Patent document 3 below). However, this developer has a tendency to damage image portions, and is insufficient to secure high printing durability. Further, as a developer containing a developer with a relatively low pH containing no silicates is disclosed an aqueous potassium hydroxide solution containing an anionic surfactant (see for example Patent document 4 below) or an aqueous alkali metal carbonate solution with a pH of from 8.5 to 11.5 (see for example Patent document 5 below). However, these relatively low pH developers have problem in that sufficient development is not obtained since solubility to a photopolymerizable light sensitive layer is poor. As a technique for overcoming this problem, a developer containing an inorganic alkali agent and a nonionic surfactant having a polyoxyethylene group is known as a developer for a planographic printing plate material comprising a photopolymerizable light sensitive layer (see for example patent documents 6 and 7 below).

However, when these developers are used over long term or are used to develop a relatively large amount of planographic printing plate materials employing an automatic developing machine, the developers may deteriorate, resulting in occurrence of contamination due to insufficient development. Further, these developers have tendency that reproduction of dot images, particularly reproduction of large dot images (with a dot area of 90 to 99%) lowers.

Patent document 1: JP-A-8-248643
Patent document 2: JP-A-11-65129
Patent document 3: JP-A-61-109052
Patent document 4: JP-A-2000-81711
Patent document 5: JP-A-11-65126
Patent document 6: JP-A-2002-196507
Patent document 7: JP-A-2002-91015

### DISCLOSURE OF THE INVENTION

### Problems to be Solved by the Invention

An object of the invention is to provide a developer and a processing method for a light sensitive printing plate material (hereinafter also referred to as a planographic printing plate material) providing excellent contamination resistance during printing and high reproduction of dot and line images.

### Means for Solving the Above Problems

The above object can be attained by one of the following constitutions.

(1) A developer for a light sensitive planographic printing plate material comprising an aluminum support and provided thereon, a light sensitive layer comprised of a light sensitive resin composition, the light sensitive planographic printing plate material after exposure being developed with the developer, wherein the developer contains a compound represented by the following formula (1),

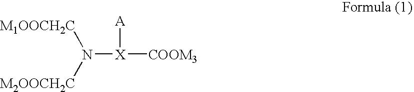

wherein A represents a substituted or unsubstituted alkyl group having a carbon atom number of from 1 to 5, a substituted or unsubstituted aryl group or COOM; M, M₁, M₂ and M₃ independently represent a hydrogen atom, an alkali metal or an ammonium group; and X represents CₘH₂ₘ₋₁ in which m represents an integer of from 1 to 5.

(2) The developer for a light sensitive planographic printing plate material of item 1 above, wherein A in formula (1) represents CH₃ or CH₂COOM₄ in which M₄ represents a hydrogen atom, an alkali metal or an ammonium group.

(3) The developer for a light sensitive planographic printing plate material of item 1 above, wherein A in formula (1) represents an alkyl group or an aryl group each having a hydroxyl group as a substituent.

(4) A method for processing a light sensitive planographic printing plate material, the method comprising the steps of developing a light sensitive planographic printing plate material with the developer of any one of items 1 through 3; and washing the developed planographic printing plate material with water.

### EFFECTS OF THE INVENTION

The present invention can provide a developer and a processing method for a light sensitive printing plate material providing excellent contamination resistance during printing and high reproduction of dot and line images.

### PREFERRED EMBODIMENT OF THE INVENTION

The present invention will be explained in detail below.

The compound represented by formula (1) will be explained below.

In formula (1) above, A represents a substituted or unsubstituted alkyl group having a carbon atom number of from 1 to 5, a substituted or unsubstituted aryl group or COOM. The substituent of the alkyl or aryl group is a halogen atom, a hydroxyl group, an amino group, a cyano group, COOM₄ or SO₃M₅, and preferably COOM₄ or a hydroxyl group. X represents a trivalent group represented by formula CₘH₂ₘ₋₁ in which m represents an integer of from 1 to 5. M, M₁, M₂, M₃, M₄ and M₅ independently represent a hydrogen atom, an alkali metal or an ammonium group. The alkali metal is preferably sodium or potassium.

Next, preferred examples of the compound represented by formula (1) will be listed, but the invention is not limited thereto.

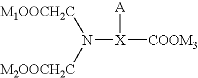

| | A | M₁ | M₂ | M₃ | X |
|---|---|---|---|---|---|
| 1-1 | —COONa | Na | Na | Na | 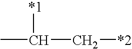 |
| 1-2 | —CH₃ | Na | Na | Na | 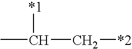 |
| 1-3 | —C₂H₅ | Na | Na | Na | 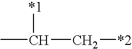 |
| 1-4 | —CH₂COOK | K | K | K | 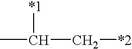 |
| 1-5 | —CH₂CH₂COOH | H | H | H | 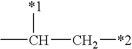 |
| 1-6 | —CH₂OH | K | K | K | 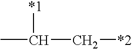 |
| 1-7 | —(CH₂)₅OH | Na | Na | Na | 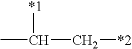 |
| 1-8 | 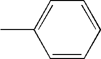 | NH₄ | NH₄ | NH₄ | 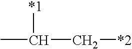 |
| 1-9 | —CH₃ | Na | Na | Na | 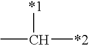 |
| 1-10 | —C₄H₉ | Na | Na | Na | 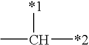 |
| 1-11 | —CH₂COONa | Na | Na | Na | 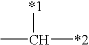 |
| 1-12 | —CH₂CH₂COONa | Na | Na | Na | 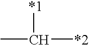 |
| 1-13 | —CH₂CH₂OH | Na | Na | Na | 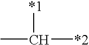 |
| 1-14 | 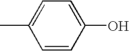 | K | K | K | 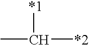 |

In the above, *1 represents a site bonding to A, and *2 represents a site bonding to —COOM₃.

The developer or developer replenisher contains preferably at least one compound selected from silic acid, phosphoric acid, carbonic acid, boric acid, phenols, saccharides, oximes, and fluorinated alcohols.

The developer or developer replenisher has a pH of preferably from more than 8.5 to less than 13.0, and more preferably from 8.5 to 12. The weak acid compounds such as phenols, saccharides, oximes, and fluorinated alcohols as described above have an acid dissociation constant (pKa) of preferably from 10.0 to 13.2. Such acid compounds are selected from those described in “IONIZATION CONSTANTS OF ORGANIC ACIDS IN AQUEOUS SOLUTION” published by Pergamon Press Co. Ltd. Examples thereof include phenols having a phenolic hydroxyl group such as salicylic acid (pKa: 13.0), 3-hydroxy-2-naphthoic acid (pKa: 12.84), catechol (pKa: 12.6), gallic acid (pKa: 12.4), sulfosalicylic acid (pKa: 11.7), 3,4-dihydroxysulfosalicylic acid (pKa: 12.2), 3,4-dihydroxybenzoic acid (pKa: 11.94), 1,2,4-trihydroxybenzene (pKa: 11.82), hydroquinone (pKa: 11.56), pyrogallol (pKa: 11.34), o-cresol (pKa: 10.33), resorcinol (pKa: 11.27), p-cresol (pKa: 10.27), and m-cresol (pKa: 10.09).

As the saccharides, non-reducing saccharides, which are stable in an alkali solution, are preferably used. The non-reducing saccharides are those which do not have a free aldehyde group or ketone group, and therefore do not have reducibility. They are classified into trehalose type oligosaccharides in which monosaccharides combine with each other through the reducing groups, glycosides in which saccharides combine with another compound through their reducing group, and sugar alcohols obtained by reducing saccharides, each of which can be suitably used in the invention. Examples of the trehalose type oligosaccharide include trehalose and saccharose. Examples of the glycosides include alkyl glycosides, phenol glycosides, and mustard oil glycoside. Examples of the sugar alcohols include D,L-arabitol, ribitol, xylitol, D,L-sorbitol, D,L-mannitol, D,L-iditol, D,L-talitol, D,L-dulcitol, and D,L-allodulcitol. Maltitol obtained by hydrogenation of disaccharides or reduced oligosaccharides (reduced starch syrup) obtained by hydrogenation of oligosaccharides are suitably used. Examples of the oximes include 2-butanoneoxime (pKa: 12.45), acetoxime (pKa: 12.42), 1,2-cycloheptanedioxime (pKa: 12.3), 2-hydroxybenzaldehideoxime (pKa: 12.10), dimethylglyoxime (pKa: 12.9), ethanediamidedioxime (pKa: 11.37), acetophenoneoxime (pKa: 11.35). Examples of the fluorinated alcohols include 2,2,3,3-tetrafluoropropanol-1 (pKa: 12.74), trifluoroethanol (pKa: 12.37), and trichloroethanol (pKa: 12.24). Further, aldehydes, nucleic acid related substances or other weak acids are used. Examples of the aldehydes include pyridine-2-aldehyde (pKa: 12.68) and pyridine-4-aldehyde (pKa: 12.05), Examples of the nucleic acid related substances include adenosine (pKa: 12.56), inosine (pKa: 12.5), guanine (pKa: 12.3), cytosine (pKa: 12.2), hypoxanthine (pKa: 12.1), and xanthine (pKa: 11.9). Examples of the other weak acids include diethylaminomethylsulfonic acid (pKa: 12.32), 1-amino-3,3,3-trifluorobenzoic acid (pKa: 12.29), isopropylidenedisulfonic acid (pKa: 12.10), 1,1-ethylidenediphosphonic acid (pKa: 11.54), 1,1-diethylidenedisulfonic acid-1-hydroxy (pKa: 11.52), benzimidazole (pKa: 12.86), thiobenzamide (pKa: 12.8), picolinthioamide (pKa: 12.55), and barbituric acid (pKa: 12.5). These acid compounds can be used singly or as a mixture of two or more thereof. Of these compounds, phosphoric acid, carbonic acid, sulfosalicylic acid, salicylic acid, sugar alcohol, and saccharose are preferred, and D-sorbitol, saccharose, and reduced starch syrup are particularly preferred, since they are inexpensive and show buffering action in appropriate pH regions.

These acid compounds are contained in the developer in an amount of preferably from 0.1 to 30% by weight, and more preferably from 1 to 20% by weight. The acid compound content less than the lower limit of the above range cannot provide sufficient buffering effect and the acid compound content more than the upper limit of the above range is difficult to obtain high concentration, resulting in cost increase. As alkali agents used in combination with the above acid compounds, sodium hydroxide, potassium hydroxide, ammonium hydroxide and lithium hydroxide are suitably used. These alkali agents may be used singly or in combination of two or more kinds thereof. The image portions of a printing plate obtained by developing a light sensitive planographic printing plate material with a developer with a pH of less than 8.5 are physically weak and likely to be worn during printing, resulting in lowering of printing durability. Further, such image portions are chemically weak, and are low in resistance to chemicals such as ink cleaning agent or a plate cleaner used during printing, resulting in poor chemical resistance. A developer with a pH exceeding 13.0 shows strong skin irritation and must be carefully handled, which is undesired.

Other examples of the alkali agents include potassium silicate, sodium silicate, lithium silicate, ammonium silicate, potassium metasilicate, sodium metasilicate, lithium metasilicate, ammonium metasilicate, potassium phosphate, sodium phosphate, lithium phosphate, ammonium phosphate, potassium hydrogenphosphate, sodium hydrogenphosphate, lithium hydrogenphosphate, ammonium hydrogenphosphate, potassium carbonate, sodium carbonate, lithium carbonate, ammonium carbonate, potassium hydrogencarbonate, sodium hydrogencarbonate, lithium hydrogencarbonate, ammonium hydrogencarbonate, potassium borate, sodium borate, lithium borate, and ammonium borate. These agents may be added in the form of salt. In order to adjust pH of the developer, sodium hydroxide, potassium hydroxide, ammonium hydroxide or lithium hydroxide can be used. Organic alkali agents such as monomethylamine, dimethylamine, trimethylamine, monoethylamine, diethylamine, triethylamine, monoisopropylamine, diisopropylamine, triisopropylamine, n-butylamine, monoethanolamine, diethanolamine, triethanolamine, monoisopropanolamine, diisopropanolamine, ethyleneimine, ethylenediamine and pyridine are also used in combination. The silicate concentration of the developer used in the invention is preferably lower, and is preferably from 0.0 to 0.5% by weight and more preferably from 0.0 to 0.1 by weight in terms of SiO₂ concentration. The developer containing no silicate is most preferable.

The developer of the invention refers to a developer (so-called working developer) to be replenished with developer replenisher in order to maintain activity of the developer which lowers during development of light sensitive planographic printing plate material, as well as fresh developer used at the beginning of development. The pH of the developer replenisher may be more than 13.0, since it is required to have higher activity (alkali concentration) than the developer.

The developer or developer replenisher in the invention can contain various surfactants or organic solvents as necessary, in order to accelerate development, disperse smuts occurring during development, or enhance ink receptivity at image portions of printing plate. Preferred examples of the nonionic surfactant include polyoxyethylenealkyl ethers, polyoxyethylenealkylphenyl ethers, polyoxyethylenepolystyrylphenyl ethers, polyoxyethylenepolyoxypropylenalkyl ethers, partial esters of glycerin and fatty acids, partial esters of sorbitan and fatty acids, partial esters of pentaerythritol and fatty acids, propylene glycol monofatty acid ester, partial esters of sucrose and fatty acids, partial esters of polyoxyethylenesorbitan and fatty acids, partial esters of polyoxyethylenesorbitol and fatty acids, esters of polyoxyethylene glycol and fatty acids, partial esters of polyglycerin and fatty acids, polyoxyethylene castor oil, partial esters of polyoxyethyleneglycerin and fatty acids, polyoxyethylene-polyoxypropylene block copolymer, adduct of polyoxyethylene-polyoxypropylene block copolymer with ethylene imine, fatty acid diethanolamides, N,N-bis-2-hydroxyalkylamines, polyoxyethylenealkylamines, triethanolamine fatty acid esters, and trialkylamine oxides. Examples of the anionic surfactant include fatty acid salts, abietic acid salts, hydroxyalkane sulfonic acid salts, alkane sulfonic acid salts, dialkylsulfosuccinic acid salts, straight-chained alkylbenzene sulfonic acid salts, branched alkylbenzene sulfonic acid salts, alkylnaphthalene sulfonic acid salts, alkyldiphenylether sulfonic acid salts, alkylphenoxypolyoxyethylenepropyl sulfonic acid salts, polyoxyethylenealkyl sulfophenylether salts, N-methyl-N-oleiltaurine sodium salts, N-alkylsulfosuccinic acid monoamide disodium salts, petroleum sulfonic acid salts, nitrated castor oil, sulfated beef tallow, fatty acid alkyl ester sulfate salts, alkylsulfate salts, polyoxyethylenealkylether sulfate ester salts, fatty acid monoglyceride sulfate ester salts, polyoxyethylenealkylphenylether sulfate ester salts, polyoxyethylenestyrylphenylether sulfate ester salts, alkylphosphate salts, polyoxyethylenealkylether phosphate ester salts, polyoxyethylenealkylphenylether phosphate ester salts, partial saponification products of styrene-maleic anhydride copolymers, partial saponification products of olefin-maleic anhydride copolymers, and condensates of naphthalene sulfonic acid salts with formalin. Examples of the cationic surfactant include alkylamine salts, quaternary ammonium salts such as tetrabutylammonium bromide, polyoxyethylene alkylamine salts, and polyethylene polyamine derivatives. Examples of the amphoteric surfactant include carboxybetains, aminn carboxylic acids, sulfobetaines, aminosulfates and imidazolines. Surfactants, in which the polyoxyethylene in the surfactants described above is replaced by polyoxypropylene or polyoxybutylene can be also used. A preferred surfactant is a fluorine-containing surfactant having a perfluoroalkyl group in the molecule. Examples thereof include aionic ones such as perfluoroalkyl carboxylic acid salts, perfluoroalkyl sulfonic acid salts, and perfluoroalkyl phosphates; amphoteric ones such as perfluoroalkyl betaines; cationic ones such as perfluoroalkyltrimethylammonium salts; and nonionic ones such as perfluoroalkylamineoxide, perfluoroalkylethylene oxide adduct, an oligomer having a perfluoroalkyl group and a hydrophilic group, an oligomer having a perfluoroalkyl group and an oleophilic group, an oligomer having a perfluoroalkyl group, a hydrophilic group and an oleophilic group, and urethanes having a perfluoroalkyl group or an oleophilic group. These surfactants may be used singly or as an admixture of two or more kinds thereof. The surfactant content of the developer is preferably from 0.001 to 10% by weight, and more preferably from 0.01 to 5% by weight.

The developer or developer replenisher preferably contains a development stabilizing agent. The preferred examples of the development stabilizing agent include an adduct of sugar alcohol with polyethylene glycol, tetraalkylammonium hydroxide such as tetrabutylammonium hydroxide, a phosphonium salt such as tetrabutylphosphonium bromide, and an iodonium salt such as diphenyliodonium chloride, as disclosed in Japanese Patent O.P.I. Publication No. 6-282079. Examples of the development stabilizing agent include anionic surfactants or amphoteric surfactants disclosed in Japanese Patent O.P.I. Publication No. 50-51324, water soluble cationic polymers disclosed in Japanese Patent O.P.I. Publication No. 55-95946, and water soluble amphoteric surfactants disclosed in Japanese Patent O.P.I. Publication No. 56-142528. Further, the examples include organic boron-containing compound to which alkylene glycol is added, disclosed in Japanese Patent O.P.I. Publication No. 59-84241, polyoxyethylene-polyoxypropylene block polymer type water-soluble surfactant, disclosed in Japanese Patent O.P.I. Publication No. 60-111264, an alkylenediamine compound having polyoxyethylene-polyoxypropylene, disclosed in Japanese Patent O.P.I. Publication No. 60-129750, polyoxyethylene, glycol with an average weight molecular weight of not less than 300 disclosed in Japanese Patent O.P.I. Publication No. 61-215554, a fluorine-containing surfactant having a cationic group disclosed in Japanese Patent O.P.I. Publication No. 63-175858, and a water soluble ethyleneoxide adduct obtained by adding ethyleneoxy to an acid or an alcohol, or water soluble polyalkylenes disclosed in Japanese Patent O.P.I. Publication No. 2-39157.

An organic solvent is optionally added to the developer or the developer replenisher. The organic solvent is a solvent having a water solubility of suitably 10 weight % or less, and preferably 5 weight % or less. Examples of the organic solvent include 1-phenylethanol, 2-phenylethanol, 3-phenyl-1-propanol, 4-phenyl-1-butanol, 1-phenyl-2-butanol, 2-phonoxyethanol, 2-benzyloxyethanol, o-methoxybenzylalcohol, m-methoxybenzylalcohol, p-methoxybenzylalcohol, benzylalcohol, cyclohexanol, 2-methylcyclohexanol, 3-methylcyclohexanol, 4-methylcyclohexanol, N-phenylethanolamine, and N-phenyldiethanolamine. The organic solvent content of the working developer is preferably 0.1 to 5 weight %. It is preferred that the organic solvent content is not substantially contained in the developer or developer replenisher. The term “not substantially contained” means that the organic solvent is contained in an amount of not more than 1% by weight.

A reducing agent is optionally added to the developer or developer replenisher used in the invention. This is added in order to prevent occurrence of stains on the printing plate during printing. The addition is particularly effective in developing a negative working light sensitive planographic printing plate precursor comprising a light sensitive diazonium compound. Preferred examples of the reducing agent include a phenolic compound such as thiosalicylic acid, hydroquinone, metol, methoxyquinone, resorcine, or 2-methylresorcine, and an amine compound such as phenylenediamine or phenylhydrazine. Preferred examples of the inorganic reducing agent include a sodium, potassium or ammonium salt of an inorganic acid such as sodium sulfite, potassium sulfite, ammonium sulfite, sodium phosphite, potassium phosphite, ammonium phosphite, sodium hydrogen phosphite, potassium hydrogen phosphite, ammonium hydrogen phosphite, sodium thiosulfate, potassium thiosulfate, ammonium thiosulfate, sodium dithionite, potassium dithionite, ammonium dithionite. It is sulfite of these that shows excellent anti-staining effect. The reducing agent content of the developer is preferably 0.05 to 5% by weight.

An organic carboxylic acid is optionally added to the developer or the developer replenisher. Preferred organic carboxylic acids include an aliphatic carboxylic acid or an aromatic carboxylic acid each having a carbon atom number of from 6 to 20. Examples of the aliphatic carboxylic acid include caproic acid, enanthic acid, caprylic acid, lauric acid, myristic acid, palmitic acid, and stearic acid. Particularly preferred are alkanoic acids having a carbon atom number of from 8 to 12. The acid may be an unsaturated acid having a double bond in the molecule or may have a branched carbon chain. The aromatic carboxylic acid is an aromatic compound such as benzene, naphthalene or anthracene having a carboxyl group. Examples of the aromatic carboxylic acid include o-chlorobenzoic acid, p-chlorobenzoic acid, o-hydroxybenzoic acid, p-hydroxybenzoic acid, o-aminobenzoic acid, p-aminobenzoic acid, 2,4-dihydroxybenzoic acid, 2,5-dihydroxybenzoic acid, 2,6-dihydroxybenzoic acid, 2,3-dihydroxybenzoic acid, 3,5-dihydroxybenzoic acid, gallic acid, 1-hydroxy-2-naphthoic acid, 3-hydroxy-2-naphthoic acid, 2-hydroxy-1-naphthoic acid, 1-naphthoic acid, and 2-naphthoic acid. Hydroxy naphthoic acids are especially preferred. These carboxylic acids are preferably used in the salt form, for example as the sodium salts, potassium salts or ammonium salts, in order to increase their water solubility. The organic carboxylic acid content of the developer is not specifically limited, but the content lass than 0.1% by weight does not exhibit advantageous effects, while the content exceeding 10% by weight cannot enhance the effects, and may prevent dissolution of other additives into the developer. Therefore, the organic carboxylic acid content of the working developer is preferably from 0.1 to 10% by weight, and more preferably from 0.5 to 4% by weight.

The developer or developer replenisher may contain the following additives in order to increase development performance. Examples of the additives include a neutral salt such as sodium chloride, potassium chloride, potassium bromide, as disclosed in Japanese Patent O.P.I. Publication No. 58-75152, a complex such as [Co(NH₃)₆]Cl₃ as disclosed in Japanese Patent O.P.I. Publication No. 59-121336, an amphoteric polymer such as a copolymer of vinylbenzyl-trimethylammonium chloride and sodium acrylate as disclosed in Japanese Patent O.P.I. Publication No. 56-142258, the organic metal containing surfactant containing Si or Ti as disclosed in Japanese Patent O.P.I. Publication No. 59-75255, and the organic boron containing compound disclosed in Japanese Patent O.P.I. Publication No. 59-84241. The developer or developer replenisher in the invention can further contain an antiseptic agent, a coloring agent, a viscosity increasing agent, an antifoaming agent, or a water softener. Examples of the antifoaming agent include mineral oil, vegetable oil, alcohols, surfactants, and silicon oil disclosed in Japanese Patent O.P.I. Publication No. 2-244143. The water softeners include polyphosphoric acid or its sodium, potassium or ammonium salt; aminopolycarboxylic acids or their salts such as ethylenediaminetetraacetic acid or its sodium, potassium or ammonium salt, diethylenetriaminepentaacetic acid or its sodium, potassium or ammonium salt, triethylenetetramine-hexaacetic acid or its sodium, potassium or ammonium salt, hydroxyethylethylenediaminetriacetic acid or its sodium, potassium or ammonium salt, nitrilotriacetic acid or its sodium, potassium or ammonium salt, 1,2-diaminocyclohexane-tetraacetic acid or its sodium, potassium or ammonium salt, 1,3-diamino-2-propanoltetraacetic acid or its sodium, potassium or ammonium salt; and phosphonic acids or their salts such as aminotri(methylenephosphonic acid) or its sodium, potassium or ammonium salt, ethylenediaminetetra-(methylenephosphonic acid) or its sodium, potassium or ammonium salt, diethylenetriaminepenta(methylenephosphonic acid) or its sodium, potassium or ammonium salt, triethylenetetraminehexa(methylenephosphonic acid) or its sodium, potassium or ammonium salt, hydroxyethylethylenediaminetri(methylenephosphonic acid) or its sodium, potassium or ammonium salt, and 1-hydroxyethane-1,1-diphosphonic acid or its sodium, potassium or ammonium salt. The water softener content of the developer varies on hardness or amount of a hard water used, but the content is preferably 0.01 to 5 weight %, and more preferably 0.01 to 0.5 weight %. The content less than the above range cannot attain the desired objects while the content exceeding the above range has an adverse effect on image areas such as dye elimination. The developer or developer replenisher is prepared by dissolving the components described above in water. The developer has a conductance of preferably from 3 to 100 mS.

The developer or developer replenisher used in the invention is an aqueous concentrated solution with a low water content, which is diluted with water and used for development. The aqueous concentrated solution is advantageous in view of its transport. The degree of concentration of the concentrated solution is such that the components contained in the solution are not separated nor precipitated. The concentrated solution may contain a solubilizing agent. As the solubilizing agent is preferred so-called a hydrotrope such as toluene sulfonic acid, xylene sulfonic acid, or their alkali metal salt, which is disclosed in Japanese Patent O.P.I. Publication Nos. 6-32081.

The developer in the form of solid or paste can be prepared by further removing water in the developer concentrate. The developer concentrate may be prepared by providing a developer solution, followed by evaporation to dryness and is preferably prepared in such a manner that plural components are mixed with a small amount of water or without adding any water. The developer concentrate can also be prepared in the form of granules or tablets, as described in Japanese Patent O.P.I. Publication Nos. 51-61837, 2-109042, 2-109043, 3-39735, 5-142786, 6-266062, and 7-13341. Materials contained in the developer concentrate are conventional ones ordinarily used in the developer for a light sensitive planographic printing plate material. It is preferred that materials, which are not reproduced by diluting the concentrate with water, are not used. For example, silicates are petrified in the absence of water and insoluble in water, and therefore, carbonates, phosphates or organic acid salts are preferably used instead of silicates.

The developer concentrate in the form of concentrated solution, solid or paste may be divided into plural parts differing in material species or compounding ratio. The concentrate is used for development preferably by diluting with water to a prescribed concentration prior to development. When the developer concentrate is used as a developer replenisher, it is preferred that the developer concentrate is diluted with water to a prescribed concentration, followed by being supplied to a working developer, whereby it is also feasible to supply a developer at a higher concentration than the prescribed one or, without being diluted to the prescribed concentration, to the working developer. When supplied at a higher concentration than the prescribed one or without being diluted to the prescribed concentration, water may be separately added in the same timing or in a different timing.

It is preferred that an automatic developing machine used in the invention is equipped with a means for automatically introducing a developer replenisher in a necessary amount into a developing bath, a means for discharging any excessive developer and a means for automatically introducing water in necessary amounts to the developing bath. It is preferred that the automatic developing machine comprises a means for detecting a planographic printing plate material to be transported, a means for calculating the area to be processed of the planographic printing plate material based on the detection, or a means for controlling a replenishing amount of a developer replenisher, a replenishing amount of water to be replenished or replenishing timing based on the detection and calculation. It is also preferred that the automatic developing machine comprises a means for controlling a temperature of a developer, a means for detecting a pH and/or electric conductivity of a developer, or a means for controlling a replenishing amount of the developer replenisher, a replenishing amount of water to be replenished and/or the replenishing timing based on the detected pH and/or electric conductivity. Where the developing step is followed by a washing step, washing water used for washing can be reused as dilution water for diluting the developer concentrate.

The automatic developing machine used in the invention may be provided with a pre-processing section to allow the plate to be immersed in a pre-processing solution prior to development. The pre-processing section is provided preferably with a mechanism of spraying a pre-processing solution onto the plate surface, preferably with a mechanism of controlling the pre-processing solution at a temperature within the range of 25 to 55° C., and preferably with a mechanism of rubbing the plate surface with a roller-type brush. Common water and the like are employed as the pre-processing solution.

The automatic developing machine used in the invention has preferably a water replenishing means for replenishing water evaporated in the developer. In the automatic developing machine having a water supply means for diluting developer concentrate, the supply means may be used as the water replenishing means. As the water replenishing method, a method is simple and preferred in which a pre-determined amount of water is replenished at a pre-determined time interval. Water replenishing amount may be corrected based on pH or electric conductivity of developer. Further, water replenishing amount may be corrected based on ambient temperature or humidity, developer temperature, or temperature or humidity in the automatic developing machine.

The planographic printing plate material developed with the developer is preferably subjected to post-processing. The post-processing comprises the step of processing the developed planographic printing plate material with a post-processing solution such as washing water, a rinsing solution containing a surfactant, a finisher or a protective gumming solution containing gum arabic or starch derivatives as a main component. The post-processing is carried out employing an appropriate combination of the post-processing solutions described above. For example, a method is preferred in which the developed planographic printing plate material is post-washed with washing water, and then processed with a rinsing solution containing a surfactant, or a developed planographic printing plate precursor is post-washed with washing water, and then processed with a finisher, since it reduces fatigue of the rinsing solution or the finisher. It is preferred that a multi-step countercurrent processing is carried out employing a rinsing solution or a finisher. The post-processing is carried out employing an automatic developing machine having a development section and a post-processing section. In the post-processing step, the developed printing plate is sprayed with the post-processing solution from a spray nozzle or is immersed into the post-processing solution in a post-processing tank. A method is known in which supplies a small amount of water onto the developed printing plate precursor to wash the precursor, and reuses the water used for washing as dilution water for developer concentrate. In the automatic developing machine, a method is applied in which each processing solution is replenished with the respective processing replenisher according to the area of the printing plate precursor to have been processed or the operating time of the machine. A method (use-and-discard method) can be applied in which the developed printing plate material is processed with fresh processing solution and discarded. The thus obtained planographic printing plate is mounted on a printing press, and printing is carried out.

Gum solution may be suitably added with acids or buffers to remove from the developed plate alkaline ingredients which are contained in the developer. Further, there may be added a hydrophilic polymer compound, a chelating agent, a lubricant, an antiseptic and a solubilizing agent. Inclusion of the hydrophilic polymer compound in the gum solution provides a function as a protecting agent to prevent the developed plate from flawing or staining.

Addition of a surfactant to the gum solution used in this invention improves the surface form of the coated layer. As the surfactant, an anionic surfactant and/or a nonionic surfactant can be used. Examples of the anionic surfactant include fatty acid salts, abietic acid salts, hydroxyalkanesulfonic acid salts, alkanesulfonic acid salts, dialkylsulfosuccinic acid salts, straight chain alkylbebzenesulfonic acid salts, branched alktlbebzenesulfonic acid salts, alkylnaphthalenesulfonic acid salts, alkylphenoxypolyoxyethylene propylsulfonic acid salts, polyoxyethylene alkylsulfophenyl ether, polyoxyethylene aryl ether sulfonic acid salts, polyoxyethylene-naphthyl ether sulfonic acid salts, N-methyl-N-oleyltaurine sodium salts, petroleum sulfonic acid salts, nitrated castor oil, sulfated tallow oil, fatty acid alkyl ester sulfuric acid ester salts, alkylnitrates, polyoxyethylene alkyl ether sulfuric acid ester salts, fatty acid monoglyceride sulfate ester salts, polyoxyethylene alkylphenyl ether sulfuric acid salts, alkylphosphate ester salts, polyoxyethylene alkyl ether phosphoric acid ester salts, polyoxyethylene alkylphenyl ether phosphoric acid ester salts, partially saponified styrene anhydrous maleic acid copolymer, partially saponified olefin-anhydrous maleic acid copolymer, and naphthalenesulfonic acid salt-formaline condensates. Of the foregoing, dialkylsulfosuccinic acid salts, alkylsulfates and alkylnaphthalenesulfonic acid salts are preferred.

Examples of the nonionic surfactant include polyoxyethylene alkyl ethers, polyoxyethylene alkylphenyl ethers, polyoxyethylene-polyoxypropylene block polymers, polyoxyethylene aryl ethers, polyoxyethylene naphthyl ethers, polyoxyethylene polystyrylphenyl ethers, polyoxyethylene polyoxypropylene alkyl ethers, glycerin fatty acid partial esters, sorbitan fatty acid partial esters, pentaerythritol fatty acid partial esters, propylene glycol monofatty acid esters, sugar fatty acid partial esters, polyoxuethylen sorbitan fatty acid partial esters, polyoxyethylene sorbitol fatty acid partial esters, polyethylene glycol fatty acid partial esters, polyglycerin fatty acid partial esters, polyoxyethylene-modified caster oils, polyoxyethylene grycerin fatty acid partial esters, fatty acid diethanolamides, N,N-bis-2-hydroxyalkylamines, polyoxyethylene alkylamine, triethanolamine fatty acid esters and trialkylamineoxides. Of the foregoing, polyoxyethylene alkylphenyl ethers and polyoxyethylene-polyoxypropylene block polymers are preferred. There are also usable fluorinated or silicone-type anionic or nonionic surfactants. The surfactants may be used as a mixture of two or more kinds thereof. For example, a combination of at least two different anionic surfactants or a combination of at least one anionic surfactant and at least one nonionic surfactant is preferred. The surfactant content is not specifically limited, but is preferably 0.01 to 20% by weight of post-processing solution.

In addition to the above ingredients, the gum solution used in the invention may contain polyhydric alcohols, alcohols or aliphatic hydrocarbons, as a lubricant.

Preferred examples of the polyhydric alcohols include ethylene glycol, diethylene glycol, triethylene glycol, propylene glycol, tetraethylene glycol, polyethylene glycol, glycerin, and sorbitol. Preferred alcohols include, for example, alkyl alcohols such as propyl alcohol, butyl alcohol, pentanol, hexanol, butanol, and octanol; and alcohols containing an aromatic ring, such as benzyl alcohol, phenoxyethanol, and phenylaminoethyl alcohol. The content of the lubricant in the gum solution is preferably from 0.1 to 50% by weight, and more preferably from 0.5 to 3.0% by weight.

There may be contained a variety of hydrophilic polymers for the purpose of enhancing film-forming ability. Any hydrophilic polymer which has been usable in a conventional gum solution is suitably usable. Examples thereof include gum Arabic, cellulose derivatives (e.g., carboxymethyl cellulose, carboxymethyl cellulose, methyl cellulose) and their modified compounds, polyvinyl alcohol and its derivatives, polyvinyl pyrrolidone, polyacrylamide and its copolymers, poly[(vinyl methyl ether)-co-(anhydrous maleic acid)], poly[(vinyl acetate)-co-(anhydrous maleic acid)], and poly[styrene-co-(anhydrous maleic acid)].

The gum solution in the invention is advantageously used within the acidic range of a pH of 3 to 6. Mineral acids, organic acids or inorganic salts are added to the post-processing solution to adjust the pH to the range of 3 to 6, preferably in an amount of 0.01 to 2% by weight. Mineral acids include, for example, nitric acid, sulfuric acid, phosphoric acid and metaphosphoric acid.

Organic acids include, for example, citric acid, acetic acid, oxalic acid, malonic acid, p-toluenesulfonic acid, tartaric acid, malic acid, lactic acid, levulinic acid, phytic acid and organic phosphonic acid. Inorganic salts include, for example, magnesium nitrate, primary phosphate, secondary phosphate, nickel sulfate, sodium hexamethanate, and sodium tripolyphosphate. Mineral acids, organic acids and inorganic salts may be used alone or as a mixture of two or more kinds thereof.

The gum solution may be added with antiseptics or defoaming agents. Examples of the antiseptics include phenol and its derivatives, formaline, imidazole derivatives, sodium dehydroacetate, 4-isothiazoline-3-one derivatives, benzoisothiazoline-3-one, benzotriazole derivatives, amidinoguanine derivatives, quaternary ammonium salts, derivatives of pyridine, quinoline and guanine, diazine, triazole derivatives, oxazole, and oxazine derivatives. A preferred content is a quantity capable of taking stable effect upon bacteria, mold or yeast, depending on the kind of bacteria, molds or yeast. The content is preferably 0.01 to 4% by weight, based on the working gum solution. Two or more antiseptic are preferably used as a mixture of two or more kinds thereof to take effects upon various kinds of bacteria or molds. Silicone defoaming agents are preferred, and any one of emulsion type and solubilization type is usable. A defoaming agent is used suitably at 0.01 to 1.0% by weight, based on the gum solution used.

Further, there may be added chelating agents. Preferred chelating agents include, for example, ethylenediaminetetraacetic acid and its sodium and potassium salts, diethylenetriaminepentaacetic acid and its sodium and potassium salts, triethylenetetraminehexaacetic acid and its sodium and potassium salts, ethylenediaminedisuccinic acid and its sodium and potassium salts, hydroxyethylethylenediaminetriacetic acid and its sodium and potassium salts, nitrilotriacetic acid and its sodium and potassium salts, and organic phosphonic acids or phosphonoalkanecarboxylic acids, such as 1-hydroxyethane-1,1-diphosphonic acid and its sodium and potassium salts, aminotri(methylenephosphonic acid) and its sodium and potassium salts. Besides the foregoing sodium and potassium salts of chelating agents, organic amine salts are also effective. Chelating agents are selected from those which can be stably present in the gum solution composition and is free from adverse effects on printing. The content thereof is preferably 0.001 to 1.0% by weight, based on the gum solution used.

In addition to the above ingredients, a lipophilicity-enhancing agent may be incorporated. Examples thereof include hydrocarbons such as turpentine oil, xylene, toluene, low heptane, solvent naphtha, kerosene, mineral spirit, petroleum fractions exhibiting a boiling point of ca. 120 to 250° C.; and plasticizers exhibiting a freezing point of 15° C. or less and a boiling point of 300° C. or more at 1 atmospheric pressure, including phthalic acid diesters such as dibutyl phthalate, diheptyl phthalate, di-n-octyl phthalate, di(2-ethylhexyl)phthalate, dinonyl phthalate, dodecyl vdilauryl phthalate, and butylbenzyl phthalate; dibasic fatty acid esters, such as dioctyl adipate, butylglycol adipate, dioctyl azelate, dibutyl sebacate, di(2-ethylhexyl)sebacate, and diocyl sebacate; epoxy-modified triglycerides such as epoxy-modified soybean oil; phosphoric acid esters such as tricresyl phosphate, trioctyl phosphate, and triscrolethyl phosphate; and benzoic acid esters such as benzyl benzoate.

Further, there are included saturated fatty acids such as caproic acid, enatoic acid, heralgonic acid, capric acid, undecylic acid, lauric acid, tridecylic acid, myristic acid, pentadecylic acid, palmitic acid, heptadecylic acid, stearic acid, nonadecanoic acid, arachic acid, behenic acid, lignoceric acid, cerotic acid, heptacosanoic acid, montanic acid, melissic acid, lacceric acid, and iso-valeric acid; and unsaturated fatty acids such as acrylic acid, crotonic acid, isocrotonic acid, undecylenic acidpleic acid, elaidic acid, cetoleic acid, nilcaic acid, btecidinic acid, sorbic acid, linolic acid, linolenic acid, arachidonic acid, propiolic acid, stearolic acid, sardine oil, tariric acid, and licanic acid. Of the foregoing, a fatty acid which is liquid at 50° C. is more preferred, one having 5 to 25 carbons is still more preferred, and one having 8 to 21 carbons is most preferred. These lipophilicity-enhancing agents may be used alone or as a mixture of two or more kinds thereof. The content thereof is preferably 0.01 to 10%, and more preferably 0.05 to 5% by weight, based on the gum solution.

The lipophilicity-enhancing agents may be incorporated through solution in the oil phase of a gum emulsion. Alternatively, they may be solubilized with the aid of a solubilizing agent.

In the invention the solid concentration of the gum solution usable in this invention is preferably 5 to 30 g/l. A gum layer thickness can be controlled by conditions of a squeezing means of a processor. The gum coverage is preferably 1 to 10 g/m². The gum coverage of more than 10 g/m² necessitates drying the plate surface at a relatively high temperature to complete drying for a short period, which is disadvantageous in terms of cost and safety, and whereby effects of this invention cannot be sufficiently achieved. A gum coverage of less than 1 g/m² results in non-uniform coating and unstable processability.

In the invention, the time from completion of coating the gum solution to start of drying is preferably 3 sec. or less, and more preferably 2 sec. or less. The shorter time enhances ink affinity.

The drying time is preferably 1 to 5 sec. Effects of this invention cannot be achieved at a drying time of more than 5 sec. A drying time of less than 1 sec. necessitates raising the plate surface temperature to sufficiently dry the lithographic printing plate, leading to disadvantages in cost and safety.

In the invention, known drying methods using a hot air heater or a far-infrared heater are applicable in this invention.

In the drying stage, solvents included in the gum solution need to be dried, necessitating securing sufficient drying temperature and heater capacity. The temperature needed for drying depends on the composition of the gum solution. In the case of the solvent of the gum solution being water, for example, the drying time is preferably 55° C. or more. The capacity is preferably at least 2.6 kW in a hot air drying system. A larger capacity is desirable and a capacity of 2.6 to 7 kW is preferred in balance with cost.

It is preferred in the invention that the planographic printing plate material is washed with a washing solution prior to development. The washing solution used prior to development is usually common water and may be a solution in which additives such as chelating agents, surfactants or antiseptics are added to water.

The chelating agents are used which are capable of coordination-bonding with a metal ion to form a chelate compound. Examples of the chelating agents include ethylenediaminetetraacetic acid and its potassium and sodium salts, ethylenediaminedisuccinic acid and its potassium and sodium salts, triethylenetetraminehexaacetic acid and its sodium and potassium salts, diethylenetriaminepentaacetic acid and its sodium and potassium salts, hydroxyethylethylenediaminetriacetic acid and its sodium and potassium salts, nitrilotriacetic acid and its sodium and potassium salts, 1-hydroxyethane-1,1-diphosphonic acid and its sodium and potassium salts, aminotri(methylenephosphonic acid) and its sodium and potassium salts and phosphonoalkanetricarboxylic acid. Besides the sodium and potassium salts of chelating agents, their organic amine salts are also effective. These chelating agents are contained in an amount of 0 to 3.0% by weight.

The surfactants include any one of anionic, nonionic, cationic and amphoteric surfactants, and anionic and nonionic surfactants are preferably used. The kind of preferred surfactants is different depending on the composition of an over-coat layer or photosensitive layer. In general are preferred surfactants which are capable of promoting dissolution of material used in the over-coat layer and exhibit less solubility for components of the photosensitive layer.

Examples of the anionic surfactants include fatty acid salts, abietic acid salts, hydroxyalkanesulfonates, alkanesulfonates, dialkylsulfosuccinates, straight chain alkylbebzenesulfonates, branched alkylbebzenesulfonates, alkylnaphthalenesulfonates, alkylphenoxypolyoxyethylene propylsulfonates, polyoxyethylene alkylsulfophenyl ether, polyoxyethylene aryl ether sulfonic acid salts, polyoxyethylene-naphthyl ether sulfonic acid salts, N-methyl-N-oleyltaurine sodium salts, petroleum sulfonic acid salts, nitrated castor oil, sulfated tallow oil, fatty acid alkyl ester sulfuric acid ester salts, alkylnitrates, polyoxyethylene alkyl ether sulfuric acid ester salts, fatty acid monoglyceride sulfate ester salts, polyoxyethylene alkylphenyl ether sulfuric acid salts, alkylphosphate ester salts, polyoxyethylene alkyl ether phosphoric acid ester salts, polyoxyethylene alkylphenyl ether phosphoric acid ester salts, partially saponified styrene anhydrous maleic acid copolymer, partially saponified olefin-anhydrous maleic acid copolymer, and naphthalenesulfonate formaline condensates. Of the foregoing, dialkylsulfosuccinates, alkylsulfates and alkylnaphthalenesulfonates are preferred.

Examples of the nonionic surfactants include polyoxyethylene alkyl ethers, polyoxyethylene alkylphenyl ethers, polyoxyethylene-polyoxypropylene block polymers, polyoxyethylene aryl ethers, polyoxyethylene naphthyl ethers, polyoxyethylene polystyrylphenyl ethers, polyoxyethylene polyoxypropylene alkyl ethers, glycerin fatty acid partial esters, sorbitan fatty acid partial esters, pentaerythritol fatty acid partial esters, propylene glycol monofatty acid esters, sugar fatty acid partial esters, polyoxuethylen sorbitan fatty acid partial esters, polyoxyethylene sorbitol fatty acid partial esters, polyethylene glycol fatty acid partial esters, polyglycerin fatty acid partial esters, polyoxyethylene-modified caster oils, polyoxyethylene grycerin fatty acid partial esters, fatty acid diethanolamides, N,N-bis-2-hydroxyalkylamines, polyoxyethylene alkylamine, triethanolamine fatty acid esters and trialkylamineoxides.

The surfactant is contained preferably in an amount of 0 to 10% by weight. The surfactant may be used in combination with defoaming agents.

The antiseptics include, for example, phenol and its derivatives, formalin, imidazole derivatives, sodium dehydroacetate, 4-isothiazoline-3-one derivatives, benzoisothiazoline-3-one, benzotriazole derivatives, amidinoguanine derivatives, quaternary ammonium salts, derivatives of pyridine, quinoline and guanine, diazine, triazole derivatives, oxazole, and oxazine derivatives. Preferred examples thereof include 1,2 benzoisothiazoline-3-one, o-phenylphenol, ethyl hydroxybenzoate, and chlorocresol.

The washing solution used prior to development is used preferably at a controlled temperature, and more preferably at 10 to 60° C. Washing can be performed using commonly known solution-feeding techniques such as a spraying, dipping or coating method. During washing, a wash promoting method employing a brush, a squeezing roll or a submerged shower in a dipping treatment can be suitably used.

Development may be conducted, immediately after the washing prior to development. Alternatively, development may be performed, after drying is conducted after the washing. The development is followed by a post-treatment such as washing, rinsing or a gumming treatment. Washing water used prior to development may also be reused as washing water or for a rinsing solution or gumming solution.

Next, the light sensitive planographic printing plate material used in the invention will be explained. The light sensitive planographic printing plate material comprises a light sensitive layer containing a photopolymerization initiator, an addition-polymerizable ethylenically unsaturated compound, a sensitizing dye and a polymer binder.

Preferred Examples of the photopolymerization initiator include a bromine-containing compound represented by formula (2) below, a titanocene compound, a monoalkyltriaryl borate compound, and an iron arene complex.

R¹—CBr₂—(C═O)—R² Formula (2)

wherein R¹ represents a hydrogen atom, a bromine atom, an alkyl group, an aryl group, an acyl group, an alkylsulfonyl group, an arylsulfonyl group or a cyano group; R² represents a monovalent substituent, provided that R¹ and R² may combine with each other to form a ring.

The compound represented by formula (2) above is preferably a compound represented by formula (3) below.

CBr₃—(C═O)—X—R³ Formula (3)

wherein R³ represents a monovalent substituent; X represents —O— or —NR⁴—, in which R⁴ represents a hydrogen atom or an alkyl group, provided that R³ and R⁴ may combine with each other to form a ring.

As the titanocene compounds, there are compounds disclosed in Japanese Patent O.P.I. Publication Nos. 63-41483 and 2-291. Preferred examples thereof include bis(cyclopentadienyl)-Ti-dichloride, bis(cyclopentadienyl)-Ti-bisphenyl, bis(cyclopentadienyl)-Ti-bis-2,3,4,5,6-pentafluorophenyl, bis(cyclopentadienyl)-Ti-bis-2,3,5,6-tetrafluorophenyl, bis(cyclopentadienyl)-Ti-bis-2,4,6-trifluorophenyl, bis(cyclopentadienyl)-Ti-bis-2,6-difluorophenyl, bis(cyclopentadienyl)-Ti-bis-2,4-difluorophenyl, bis(methylcyclopentadienyl)-Ti-bis-2,3,4,5,6-pentafluorophenyl, bis(methylcyclopentadienyl)-Ti-bis-2,3,5,6-tetrafluorophenyl, bis(methylcyclopentadienyl)-Ti-bis-2,4-difluorophenyl (IRUGACURE 727L, produced by Ciba Specialty Co., Ltd.), bis(cyclopentadienyl)-bis(2,6-difluoro-3-(pyry-1-yl)phenyl) titanium (IRUGACURE 784, produced by Ciba Specialty Co., Ltd.), bis(cyclopentadienyl)-bis(2,4,6-trifluoro-3-(pyry-1-yl)phenyl) titanium, and bis (cyclopentadienyl)-bis(2,4,6-trifluoro-3-(2,5-dimethylpyry-1-yl)phenyl) titanium.

As the monoalkyltriaryl borate compounds, there are those described in Japanese Patent O.P.I. Publication Nos. 62-150242 and 62-143044. Preferred examples of the monoalkyltriaryl borate compounds include tetra-n-butyl ammonium n-butyl-trinaphthalene-1-yl-borate, tetra-n-butyl ammonium n-butyl-triphenyl-borate, tetra-n-butyl ammonium n-butyl-tri-(4-tert-butylphenyl)-borate, tetra-n-butyl ammonium n-hexyl-tri-(3-chloro-4-methylphenyl)-borate, and tetra-n-butyl ammonium n-hexyl-tri-(3-fluorophenyl)-borate.

As the iron arene complexes, there are those described in Japanese Patent O.P.I. Publication No. 59-219307. Preferred examples of the iron arene complex include η-benzene-(η-cyclopentadienyl)iron•hexafluorophosphate, η-cumene)-(η-cyclopentadienyl)iron•hexafluorophosphate, η-fluorene-(η-cyclopentadienyl)iron•hexafluorophosphate, η-naphthalene-(η-cyclopentadienyl)iron•hexafluorophosphate, η-xylene-(η-cyclopentadienyl)iron•hexafluorophosphate, and η-benzene-(η-cyclopentadienyl)iron•hexafluoroborate.

Another photopolymerization initiator can be used in combination. Examples thereof include carbonyl compounds, organic sulfur compounds, peroxides, redox compounds, azo or diazo compounds, halides and photo-reducing dyes disclosed in J. Kosar, “Light Sensitive Systems”, Paragraph 5, and those disclosed in British Patent No. 1,459,563.

Typical examples of the photopolymerization initiator used in combination include the following compounds:

A benzoin derivative such as benzoin methyl ether, benzoin i-propyl ether, or α,α-dimethoxy-α-phenylacetophenone; a benzophenone derivative such as benzophenone, 2,4-dichlorobenzophenone, o-benzoyl methyl benzoate, or 4,4′-bis(dimethylamino) benzophenone; a thioxanthone derivative such as 2-chlorothioxanthone, 2-i-propylthioxanthone; an anthraquinone derivative such as 2-chloroanthraquinone or 2-methylanthraquinone; an acridone derivative such as N-methylacridone or N-butylacridone; α,α-diethoxyacetophenone; benzil; fluorenone; xanthone; an uranyl compound; a triazine derivative disclosed in Japanese Patent Publication Nos. 59-1281 and 61-9621 and Japanese Patent O.P.I. Publication No. 60-60104; an organic peroxide compound disclosed in Japanese Patent O.P.I. Publication Nos. 59-1504 and 61-243807; a diazonium compound in Japanese Patent Publication Nos. 43-23684, 44-6413, 47-1604 and U.S. Pat. No. 3,567,453; an organic azide compound disclosed in U.S. Pat. Nos. 2,848,328, 2,852,379 and 2,940,853; orthoquinondiazide compounds disclosed in Japanese Patent Publication Nos. 36-22062b, 37-13109, 38-18015 and 45-9610; various onium compounds disclosed in Japanese Patent Publication No. 55-39162, Japanese Patent O.P.I. Publication No. 59-14023 and “Macromolecules”, Volume 10, p. 1307 (1977); azo compounds disclosed in Japanese Patent Publication No. 59-142205; metal arene complexes disclosed in Japanese Patent O.P.I. Publication No. 1-54440, European Patent Nos. 109,851 and 126,712, and “Journal of Imaging Science”, Volume 30, p. 174 (1986); (oxo) sulfonium organoboron complexes disclosed in Japanese Patent O.P.I. Publication Nos. 5-213861 and 5-255347; titanocenes disclosed in Japanese Patent O.P.I. Publication Nos. 59-152396 and 61-151197; transition metal complexes containing a transition metal such as ruthenium disclosed in “Coordination Chemistry Review”, Volume 84, p. 85-277 (1988) and Japanese Patent O.P.I. Publication No. 2-182701; 2,4,5-triarylimidazol dimmer disclosed in Japanese Patent O.P.I. Publication No. 3-209477; carbon tetrabromide; organic halide compounds disclosed in Japanese Patent O.P.I. Publication No. 59-107344.

When a laser is used as a light source for exposure, the light sensitive layer preferably contains a sensitizing dye. As the sensitizing dye, a sensitizing dye is preferred which has an absorption maximum in the wavelength or its vicinity of light emitted from the light source.

Examples of the sensitizing dyes, which have sensitivity in the wavelengths of visible to near infrared regions, include cyanines, phthalocyanines, merocyanines, porphyrins, spiro compounds, ferrocenes, fluorenes, fulgides, imidazoles, perylenes, phenazines, phenothiazines, polyenes, azo compounds, diphenylmethanes, triphenylmethanes, polymethine acridines, cumarines, ketocumarines, quinacridones, indigos, styryl dyes, pyrylium dyes, pyrromethene dyes, pyrazolotriazole compounds, benzothiazole compounds, barbituric acid derivatives, thiobarbituric acid derivatives, ketoalcohol borate complexes, and compounds disclosed in European Patent No. 568,993, U.S. Pat. Nos. 4,508,811 and 5,227,227, and Japanese Patent O.P.I. Publication Nos. 2001-125255 and 11-271969.

Examples in which the above polymerization initiators are used in combination with the sensitizing dye are disclosed in Japanese Patent O.P.I. Publication Nos. 2001-125255 and 11-271969.

The photopolymerization initiator content of the light sensitive layer is not specifically limited, but is preferably from 0.2 to 20 parts by weight based on the 100 parts by weight of polymerizable compound or cross-linkable compound. The content ratio by mole of the photopolymerization initiator to the sensitizing dye is preferably from 1:100 to 100:1.

As the addition-polymerizable ethylenically unsaturated compound in the invention, there are known monomers such as a radically polymerizable monomer, and a polyfunctional monomer or oligomer having two or more of an ethylenic double bond in the molecule generally used in an ultraviolet curable resin composition. The monomers are not specifically limited.

Preferred examples thereof include a monofunctional acrylate such as 2-ethylhexyl acrylate, 2-hydroxypropyl acrylate, glycerol acrylate, tetrahydrofurfuryl acrylate, phenoxyethyl acrylate, nonylphenoxyethyl acrylate, tetrahydrofurfuryl-oxyethyl acrylate, tetrahydrofurfuryloxyhexanorideacrylate, an ester of 1,3-dioxane-ε-caprolactone adduct with acrylic acid, or 1,3-dioxolane acrylate; a methacrylate, itaconate, crotonate or maleate alternative of the above acrylate; a bifunctional acrylate such as ethyleneglycol diacrylate, triethyleneglycol diacrylate, pentaerythritol diacrylate, hydroquinone diacrylate, resorcin diacrylate, hexanediol diacrylate, neopentyl glycol diacrylate, tripropylene glycol diacrylate, hydroxypivalic acid neopentyl glycol diacrylate, neopentyl glycol adipate diacrylate, diacrylate of hydroxypivalic acid neopentyl glycol-ε-caprolactone adduct, 2-(2-hydroxy-1,1-dimethylethyl)-5-hydroxymethyl-5-ethyl-1,3-dioxane diacrylate, tricyclodecanedimethylol acrylate, tricyclodecanedimethylol acrylate-ε-caprolactone adduct or 1,6-hexanediol diglycidylether diacrylate; a dimethacrylate, diitaconate, dicrotonate or dimaleate alternative of the above diacrylate; a polyfunctional acrylate such as trimethylolpropane triacrylate, ditrimethylolpropane tetraacrylate, trimethylolethane triacrylate, pentaerythritol triacrylate, pentaerythritol tetraacrylate, dipentaerythritol tetraacrylate, dipentaerythritol pentaacrylate, dipentaerythritol hexacrylate, dipentaerythritol hexacrylate-ε-caprolactone adduct, pyrrogallol triacrylate, propionic acid dipentaerythritol triacrylate, propionic acid dipentaerythritol tetraacrylate or hydroxypivalylaldehyde modified dimethylolpropane triacrylate; a methacrylate, itaconate, crotonate or maleate alternative of the above polyfunctional acrylate.

A prepolymer can be used as the ethylenically unsaturated compound, and examples of the prepolymer include compounds as described later. The prepolymer with a photopolymerizable property, which is obtained by incorporating acrylic acid or methacrylic in an oligomer with an appropriate molecular weight, can be suitably employed. These prepolymers can be used singly, in combination or as their mixture with the above described monomers and/or oligomers.

Examples of the prepolymer include polyester (meth)acrylate obtained by incorporating (meth)acrylic acid in a polyester of a polybasic acid such as adipic acid, trimellitic acid, maleic acid, phthalic acid, terephthalic acid, hymic acid, malonic acid, succinic acid, glutaric acid, itaconic acid, pyromellitic acid, fumalic acid, pimelic acid, sebatic acid, dodecanic acid or tetrahydrophthalic acid with a polyol such as ethylene glycol, ethylene glycol, diethylene glycol, propylene oxide, 1,4-butane diol, triethylene glycol, tetraethylene glycol, polyethylene glycol, grycerin, trimethylol propane, pentaerythritol, sorbitol, 1,6-hexanediol or 1,2,6-hexanetriol; an epoxyacrylate such as bisphenol A•epichlorhydrin•(meth)acrylic acid or phenol novolak•epichlorhydrin•(meth)acrylic acid obtained by incorporating (meth)acrylic acid in an epoxy resin; an urethaneacrylate such as ethylene glycol•adipic acid•tolylenediisocyanate•2-hydroxyethylacrylate, polyethylene glycol•tolylenediisocyanate•2-hydroxyethylacrylate, hydroxyethylphthalyl methacrylate•xylenediisocyanate, 1,2-polybutadieneglycol•tolylenediisocyanate•2-hydroxyethylacrylate or trimethylolpropane•propylene glycol•tolylenediisocyanate•2-hydroxyethylacrylate, obtained by incorporating (meth)acrylic acid in an urethane resin; a silicone acrylate such as polysiloxane acrylate, or polysiloxane•diisocyanate•2-hydroxyethylacrylate; an alkyd modified acrylate obtained by incorporating a methacroyl group in an oil modified alkyd resin; and a spiran resin acrylate.

The light sensitive layer in the invention may contain a monomer such as a phosphazene monomer, triethylene glycol, an EO modified isocyanuric acid diacrylate, an EO modified isocyanuric acid triacrylate, dimethyloltricyclodecane diacrylate, trimethylolpropane acrylate benzoate, an alkylene glycol acrylate, or a urethane modified acrylate, or an addition polymerizable oligomer or prepolymer having a structural unit derived from the above monomer.

The ethylenic monomer used in combination is preferably a phosphate compound having at least one (meth)acryloyl group. The phosphate compound is a compound having a (meth)acryloyl group in which at least one hydroxyl group of phosphoric acid is esterified, and the phosphate compound is not limited as long as it has a (meth)acryloyl group.

Besides the above compounds, compounds disclosed in Japanese Patent O.P.I. Publication Nos. 58-212994, 61-6649, 62-46688, 62-48589, 62-173295, 62-187092, 63-67189, and 1-244891, compounds described on pages 286 to 294 of “11290 Chemical Compounds” edited by Kagakukogyo Nipposha, and compounds described on pages 11 to 65 of “UV•EB Koka Handbook (Materials)” edited by Kobunshi Kankokai can be suitably used. Of these compounds, compounds having two or more acryl or methacryl groups in the molecule are preferable, and those having a molecular weight of not more than 10,000, and preferably not more than 5,000 are more preferable.

In the invention, an ethylenically unsaturated monomer having a tertiary amino group in the molecule is preferably used. Its molecular structure is not limited, but those are preferred in which a tertiary amine having a hydroxyl group is modified with glycidyl methacrylate, methacrylic chloride, or acrylic chloride. Examples thereof include a polymerizable compound disclosed in Japanese Patent O.P.I. Publication Nos. 1-165613, 1-203413 and 1-197213.

A reaction product of a polyhydric alcohol having (a) a tertiary amino group in the molecule, (b) a diisocyanate and (c) a compound having both hydroxyl group and ethylenically double bond in the molecule is more preferably used in the invention.

Examples of the polyhydric alcohol having a tertiary amino group in the molecule include triethanolamine, N-methyldiethanolamine, N-ethyldiethanolamine, N-ethyldiethanolamine, N-n-butyldiethanolamine, N-tert-butyldiethanolamine, N,N-di(hydroxyethyl)aniline, N,N,N′,N′-tetra-2-hydroxypropylethylenediamine, p-tolyldiethanolamine, N,N,N′,N′-tetra-2-hydroxyethylethylenediamine, N,N-bis(2-hydroxypropyl)aniline, allyldiethanolamine, 3-dimethylamino-1,2-propane diol, 3-diethylamino-1,2-propane diol, N,N-di(n-propylamino)-2,3-propane diol, N,N-di(iso-propylamino)-2,3-propane diol, and 3-(N-methyl-N-benzylamino)-1,2-propane diol, but the invention is not specifically limited thereto.

Examples of the diisocyanate include butane-1,4-diisocyanate, hexane-1,6-diisocyanate, 2-methylpentane-1,5-diisocyanate, octane-1,8-diisocyanate, 1,3-diisocyanatomethylcyclohexanone, 2,2,4-trimethylhexane-1,6-diisocyanate, isophorone diisocyanate, 1,2-phenylene diisocyanate, 1,3-phenylene diisocyanate, 1,4-phenylene diisocyanate, tolylene-2,4-diisocyanate, tolylene-2,5-diisocyanate, tolylene-2,6-diisocyanate, 1,3-di(isocyanatomethyl)benzene, and 1,3-bis(1-isocyanato-1-methylethyl)benzene, but the invention is not specifically limited thereto. Examples of the compound having both hydroxyl group and ethylenically double bond in the molecule include 2-hydroxyethyl methacrylate, 2-hydroxyethyl acrylate, 4-hydroxybutyl acrylate, 2-hydroxypropylene-1,3-dimethacrylate, and 2-hydroxypropylene-1-methacrylate-3-acrylate.

The reaction product above can be synthesized according to the same method as a conventional method in which a urethaneacrylate compound is ordinarily synthesized employing an ordinary diol, a diisocyanate and an acrylate having a hydroxyl group.

Examples of the reaction product of a polyhydric alcohol having a tertiary amino group in the molecule, a diisocyanate and a compound having a hydroxyl group and an ethylenically double bond in the molecule will be listed below.

M-1: A reaction product of triethanolamine (1 mole), hexane-1,6-diisocyanate (3 moles), and 2-hydroxyethyl methacrylate (3 moles)
M-2: A reaction product of triethanolamine (1 mole), isophorone diisocyanate (3 moles), and 2-hydroxyethyl methacrylate (3 moles)
M-3: A reaction product of N-n-butyldiethanolamine (1 mole), 1,3-bis(1-cyanato-1-methylethyl)benzene (2 moles), and 2-hydroxypropylene-1-methacrylate-3-acrylate (2 moles)
M-4: A reaction product of N-n-butyldiethanolamine (1 mole), 1,3-di(cyanatomethyl)benzene (2 moles), and 2-hydroxypropylene-1-methacrylate-3-acrylate (2 moles)
M-5: A reaction product of N-methydiethanolamine (1 mole), tolylene-2,4-diisocyanate (2 moles), and 2-hydroxypropylene-1,3-dimethacrylate (2 moles)

In addition to the above, acrylates or methacrylates disclosed in Japanese Patent O.P.I. Publication Nos. 1-105238 and 2-127404 can be used.

Examples of the polymer binder used in the light sensitive layer in the invention include a polyacrylate resin, a polyvinylbutyral resin, a polyurethane resin, a polyamide resin, a polyester resin, an epoxy resin, a phenol resin, a polycarbonate resin, a polyvinyl butyral resin, a polyvinyl formal resin, a shellac resin, or another natural resin can be used. These resins can be used as an admixture of two or more thereof.

The polymer binder used in the invention is preferably a vinyl copolymer obtained by copolymerization of an acryl monomer, and more preferably a copolymer containing (a) a carboxyl group-containing monomer unit and (b) an alkyl methacrylate or alkyl acrylate unit as the copolymerization component.

Examples of the carboxyl group-containing monomer include an α,β-unsaturated carboxylic acid, for example, acrylic acid, methacrylic acid, maleic acid, maleic anhydride, itaconic acid, itaconic anhydride or a carboxylic acid such as a half ester of phthalic acid with 2-hydroxymethacrylic acid.

Examples of the alkyl methacrylate or alkyl acrylate include an unsubstituted alkyl ester such as methylmethacrylate, ethylmethacrylate, propylmethacrylate, butylmethacrylate, amylmethacrylate, hexylmethacrylate, heptylmethacrylate, octylmethacrylate, nonylmethacrylate, decylmethacrylate, undecylmethacrylate, dodecylmethacrylate, methylacrylate, ethylacrylate, propylacrylate, butylacrylate, amylacrylate, hexylacrylate, heptylacrylate, octylacrylate, nonylacrylate, decylacrylate, undecylacrylate, or dodecylacrylate; a cyclic alkyl ester such as cyclohexyl methacrylate or cyclohexyl acrylate; and a substituted alkyl ester such as benzyl methacrylate, 2-chloroethyl methacrylate, N,N-dimethylaminoethyl methacrylate, glycidyl methacrylate, benzyl acrylate, 2-chloroethyl acrylate, N,N-dimethylaminoethyl acrylate or glycidyl acrylate.

The polymer binder in the invention can further contain, as another monomer unit, a monomer unit derived from the monomer described in the following items (1) through (14):

(1) A monomer having an aromatic hydroxy group, for example, o-, (p- or m-) hydroxystyrene, or o-, (p- or m-) hydroxyphenylacrylate;

(2) A monomer having an aliphatic hydroxy group, for example, 2-hydroxyethyl acrylate, 2-hydroxyethyl methacrylate, N-methylolacrylamide, N-methylolmethacrylamide, 4-hydroxybutyl acrylate, 4-hydroxybutyl methacrylate, 5-hydroxypentyl acrylate, 5-hydroxypentyl methacrylate, 6-hydroxyhexyl acrylate, 6-hydroxyhexyl methacrylate, N-(2-hydroxyethyl)acrylamide, N-(2-hydroxyethyl)methacrylamide, or hydroxyethyl vinyl ether;

(3) A monomer having an aminosulfonyl group, for example, m- or p-aminosulfonylphenyl methacrylate, m- or p-aminosulfonylphenyl acrylate, N-(p-aminosulfonylphenyl)methacrylamide, or N-(p-aminosulfonylphenyl)acrylamide;

(4) A monomer having a sulfonamido group, for example, N-(p-toluenesulfonyl)acrylamide, or N-(p-toluenesulfonyl)methacrylamide;

(5) An acrylamide or methacrylamide, for example, acrylamide, methacrylamide, N-ethylacrylamide, N-hexylacrylamide, N-cyclohexylacrylamide, N-phenylacrylamide, N-nitrophenylacrylamide, N-ethyl-N-phenylacrylamide, N-4-hydroxyphenylacrylamide, or N-4-hydroxyphenylmethacrylamide;

(6) A monomer having a fluorinated alkyl group, for example, trifluoromethyl acrylate, trifluoromethyl methacrylate, tetrafluoropropyl methacrylate, hexafluoropropyl methacrylate, octafluoropentyl acrylate, octafluoropentyl methacrylate, heptadecafluorodecyl methacrylate, heptadecafluorodecyl methacrylate, or N-butyl-N-(2-acryloxyethyl)heptadecafluorooctylsulfonamide;

(7) A vinyl ether, for example, ethyl vinyl ether, 2-chloroethyl vinyl ether, propyl vinyl ether, butyl vinyl ether, octyl vinyl ether, or phenyl vinyl ether;

(8) A vinyl ester, for example, vinyl acetate, vinyl chroloacetate, vinyl butyrate, or vinyl benzoate;

(9) A styrene, for example, styrene, methylstyrene, or chloromethystyrene;

(10) A vinyl ketone, for example, methyl vinyl ketone, ethyl vinyl ketone, propyl vinyl ketone, or phenyl vinyl ketone;

(11) An olefin, for example, ethylene, propylene, isobutylene, butadiene, or isoprene;

(12) N-vinylpyrrolidone, N-vinylcarbazole, or N-vinylpyridine,

(13) A monomer having a cyano group, for example, acrylonitrile, methacrylonitrile, 2-pentenenitrile, 2-methyl-3-butene nitrile, 2-cyanoethyl acrylate, or o-, m- or p-cyanostyrene;

(14) A monomer having an amino group, for example, N,N-diethylaminoethyl methacrylate, N,N-dimethylaminoethyl acrylate, N,N-dimethylaminoethyl methacrylate, polybutadiene urethane acrylate, N,N-dimethylaminopropyl acrylamide, N,N-dimethylacrylamide, acryloylmorpholine, N-isopropylacrylamide, or N,N-diethylacrylamide.

Further another monomer may be copolymerized with the above monomer.

As the polymer binder is also preferred an unsaturated bond-containing copolymer which is obtained by reacting a carboxyl group contained in the above vinyl copolymer molecule with for example, a compound having a (meth)acryloyl group and an epoxy group.

Examples of the compound having a (meth)acryloyl group and an epoxy group in the molecule include glycidyl acrylate, glycidyl methacrylate and an epoxy group-containing unsaturated compound disclosed in Japanese Patent O.P.I. Publication No. 11-27196.

The weight average molecular weight of the above copolymer is preferably 10,000 to 200,000 measured by gel permeation chromatography (GPC), but is not limited thereto.

The polymer binder content of the light sensitive layer is preferably from 10 to 90% by weight, more preferably from 15 to 70% by weight, and still more preferably from 20 to 50% by weight, in view of sensitivity.

The polymer binder in the invention has an acid value of preferably from 10 to 150, more preferably from 30 to 120, and still more preferably from 50 to 90, in obtaining an optimum polarity of the light sensitive layer. The above acid value range can prevent aggregation of pigments of a light sensitive layer coating liquid.

The light sensitive planographic printing plate material in the invention preferably contains an oxygen-shielding layer. A water-soluble polymer capable of forming a layer having a low oxygen transmittance is used in the oxygen-shielding layer. Polyvinyl alcohol and/or polyvinyl pyrrolidone are preferably used in the oxygen-shielding layer. Polyvinyl alcohol has the effect of preventing oxygen from transmitting and polyvinyl pyrrolidone has the effect of increasing adhesion between the oxygen-shielding layer and the photopolymerizable light sensitive layer adjacent thereto.

Besides the above two polymers, the oxygen-shielding layer may contain a water soluble polymer such as polysaccharide, polyethylene glycol, gelatin, glue, casein, hydroxyethyl cellulose, carboxymethyl cellulose, methyl cellulose, hydroxyethyl starch, gum arabic, sucrose octacetate, ammonium alginate, sodium alginate, polyvinyl amine, polyethylene oxide, polystyrene sulfonic acid, polyacrylic acid, or a water soluble polyamide.

The planographic printing plate material in the invention preferably comprises an over coat layer as the outermost layer. The adhesive strength between the over coat layer and the light sensitive layer is preferably not less than 35 g/10 mm, more preferably not less than 50 g/10 mm, and still more preferably not less than 75 g/10 mm. Preferred composition of the overcoat layer is disclosed in Japanese Patent O.P.I. Publication No. 10-10742.

The adhesive strength can be determined according to the following method. The adhesive tape with a sufficient adhesive force is applied on the over coat layer, and then peeled together with the overcoat layer under the applied tape in the normal direction relative to the over coat layer surface. Force necessary to peel the tape together with the overcoat layer is defined as adhesive strength.

The oxygen shielding layer may further contain a surfactant or a matting agent. The oxygen shielding layer is formed, coating on the photopolymerizable light sensitive layer a coating solution in which the components described above overcoat layer are dissolved in an appropriate coating solvent, and drying. The main solvent of the coating solution is preferably water or an alcohol solvent such as methanol, ethanol, or iso-propanol.

The thickness of the oxygen shielding layer is preferably 0.1 to 5.0 μm, and more preferably 0.5 to 3.0 μm.

In the invention, there may further be combined printing plate materials, plate surface-protecting agents, raw materials for developer and automatic developing machines, as described in, for example, JP-A Nos. 11-065129, 11-065126, 2000-206706, 2000-081711, 2002-091014, 2002-091015, 2002-091016, 2002-091917, 2002-174907, 2002-182401, 2002-196506, 2002-196507, 2001-202616, 2002-229187, 2002-202615, 2002-251019, 2002-365813, 2003-029427, 2003-021908, 2003-015318, 2003-035960, 2003-043693, 2003-043701, 2003-043702 and 2003-043703.

### EXAMPLES

Next, the present invention will be explained employing a synthetic example, a support manufacturing example and examples, but the present invention is not limited thereto. In the examples, “parts” represents “parts by weight”, unless otherwise specified.

### (Synthesis of Binder)

Twelve parts of methacrylic acid, 70 parts of methyl methacrylate, 8 parts of acrylonitrile, 10 parts of ethyl methacrylate, 500 parts of ethanol, and 3 parts of α,α′-azobisisobutylonitrile were put in a three neck flask under nitrogen atmosphere, and reacted under nitrogen-atmosphere for 6 hours at 80° C. in an oil bath. After that, 3 parts of triethylammonium chloride and 2 parts of glycidyl methacrylate were added to the resulting reaction mixture, and further reacted for 3 hours. Thus, acryl copolymer 1 was obtained. The weight average molecular weight of the copolymer 1 was 50,000, measured according to GPC. The glass transition temperature Tg of the copolymer 1 was 85° C., measured according to DSC (differential thermal analysis).

### (Preparation of Support)

A 0.24 mm thick aluminum plate (material 1050, quality H16) was degreased at 60° C. for one minute in a 5% sodium hydroxide solution, washed with water, immersed at 25° C. for one minute in a 10% hydrochloric acid solution to neutralize, and then washed with water. The resulting aluminum plate was electrolytically etched using an alternating current at 25° C. for 60 seconds at a current density of 100 A/dm² in a 0.3 weight % nitric acid solution, and desmut at 60° C. for 10 seconds in a 5% sodium hydroxide solution. The desmut aluminum plate was anodized at 25° C. for 1 minute at a current density of 10 A/dm² and at a voltage of 15 V in a 15% sulfuric acid solution, and subjected to sealing treatment in a 90° C. 3% sodium silicate solution. Thus, support was obtained. The center line average surface roughness (Ra) of the support was 0.65 μm.

### (Preparation of Subbed Support)

The following subbing layer coating liquid was coated on the support obtained above using a wire bar, dried at 90° C. for one minute, and further heated at 110° C. for 3 minutes to give a subbing layer with a dry thickness of 0.1 g/m². Thus, subbed support was obtained.

(Subbing layer coating liquid)

| | | |
|---|---|---|
| γ-Methacryloxypropyltrimethoxysilane | 1 | part |
| Methyl ethyl ketone | 80 | parts |
| Cyclohexanone | 19 | parts |

(Preparation of light sensitive planographic printing plate material)

The following photopolymerizable light sensitive layer coating liquid was coated on the subbed support using a wire bar, and dried at 95° C. for 1.5 minutes to give a light sensitive layer with a dry thickness of 1.4 g/m². After that, the following over coat layer coating liquid was coated on the light sensitive layer of the resulting plate using an applicator, and dried at 75° C. for 1.5 minutes to obtain an over coat layer with a dry thickness of 2.0 g/m². Thus, a light sensitive planographic printing plate material sample having the over coat layer-provided on the light sensitive layer was prepared.

Photopolymerizable light sensitive layer coating liquid

| | |
|---|---|
| Acryl copolymer 1 | 35.0 parts |
| (Synthetic binder with a molecular weight Mw of 50,000) | |
| Spectral sensitizing dye 1 | 2.0 parts |
| Spectral sensitizing dye 2 | 2.0 parts |
| IRGACURE 784 (produced by | 4.0 parts |
| Ciba Speciality Co., Ltd.) | |
| EO-modified tris(acryloxyethyl)isocyanuric | 35.0 parts |
| Acid (ARONIX M-315, produced by Toa Gosei Co., Ltd.) | |
| Polytetramethylene glycol diacrylate | 10.0 parts |
| (PTMGA-250, produced by Kyoeisha Chemical Co., Ltd.) | |
| Polyfunctional urethane acrylate | 5.0 parts |
| (U-15HA, produced by ShinNakamura | |
| Kagaku Kogyo Co., Ltd.) | |
| Phthalocyanine pigment | 6.0 parts |
| (MHI 454 produced by Mikuni Sikisosha) | |
| 2-t-butyl-6-(3-t-butyl-2-hydroxy-5-methylbenzyl)- | 0.5 parts |
| 4-methylphenyl acrylate | |
| (Sumirizer GS produced by Sumitomo 3M Co., Ltd.) | |
| Fluorine-contained surfactant | 0.5 parts |
| (FC-431 produced by Sumitomo 3M Co., Ltd.) | |
| Methyl ethyl ketone (bp. 79.6° C.) | 80 parts |
| Cyclopentanone (bp. 129° C.) | 820 parts |
| Spectral sensitizing dye 1 | |
| 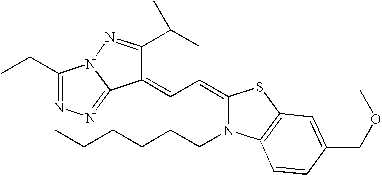 | |
| Spectral sensitizing dye 2 | |
| 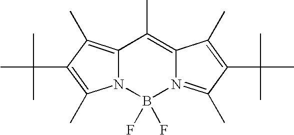 | |
| Over coat layer coating liquid | |
| Polyvinyl alcohol | 89 parts |
| (GL-03 produced by Nippon Gosei Kagaku Co., Ltd.) | |
| Water-soluble polyamide | 10 parts |
| (P-70 produced by Toray Co., Ltd.) | |
| Surfactant | 0.5 parts |
| (F142D produced by Dainippon Ink Kagaku | |
| Kogyo Co., Ltd.) | |
| Water | 900 parts |

The thus obtained light sensitive planographic printing plate material was subjected to image formation processing as described later. During processing, the following developer and gum solution were employed.

(Developer composition)

| Developer 1 | | |
|---|---|---|
| Polyoxyethylene (13) naphthyl ether | 20.0 | g/liter |
| Polyoxyethylene (13) naphthyl ether | 20 | g/liter |
| sulfonic acid ester sodium salt | | |
| Compound represented by formula (1) | 1.0 | g/liter |
| (as shown in Table 1) | | |
| Ethylenediamine disuccinic acid trisodium salt | 0.2 | g/liter |
| Potassium hydroxide | Amount giving pH 12.0 | |
| Water | Amount making 1 | liter |
| Developer replenisher 1 | | |
| Polyoxyethylene (13) naphthyl ether | 20.0 | g/liter |
| Polyoxyethylene (13) naphthyl ether | 20 | g/liter |
| sulfonic acid ester sodium salt | | |
| Compound represented by formula (1) | 1.0 | g/liter |
| (as shown in Table 1) | | |
| Ethylenediamine disuccinic acid trisodium salt | 0.2 | g/liter |
| Potassium hydroxide | Amount giving pH 12.8 | |
| Water | Amount making 1 | liter |
| Developer 2 | | |
| Potassium carbonate | 2.5 | g/liter |
| Potassium hydrogen carbonate | 5.0 | g/liter |
| Polyoxyethylene (10) benzyl ether | 50.0 | g/liter |
| Compound represented by formula (1) | 1.0 | g/liter |
| (as shown in Table 1) | | |
| Ethylenediamine disuccinic acid trisodium salt | 0.2 | g/liter |
| Potassium hydroxide | Amount giving pH 11.4 | |
| Water | Amount making 1 | liter |
| Developer replenisher 2 | | |
| Potassium carbonate | 2.5 | g/liter |
| Potassium hydrogen carbonate | 5.0 | g/liter |
| Polyoxyethylene (10) benzyl ether | 50.0 | g/liter |
| Compound represented by formula (1) | 1.0 | g/liter |
| (as shown in Table 1) | | |
| Ethylenediamine disuccinic acid trisodium salt | 0.2 | g/liter |
| Potassium hydroxide | Amount giving pH 12.2 | |
| Water | Amount making 1 | liter |
| Developer 3 | | |
| Potassium silicate solution | 40.0 | g/liter |
| (containing 26% by weight of SiO₂ and | | |
| 13.5% by weight of K₂O) | | |
| Compound represented by formula (1) | 1.0 | g/liter |
| (as shown in Table 1) | | |
| Ethylenediamine tetraacetic acid disodium salt | 0.2 | g/liter |
| Polyoxyethylene (13) naphthyl ether | 40.0 | g/liter |
| Potassium hydroxide | Amount giving pH 12.3 | |
| Water | Amount making 1 | liter |
| Developer replenisher 3 | | |
| Potassium silicate solution | 40.0 | g/liter |
| (containing 26% by weight of SiO₂ and | | |
| 13.5% by weight of K₂O) | | |
| Compound represented by formula (1) | 1.0 | g/liter |
| (as shown in Table 1) | | |
| Ethylenediamine tetraacetic acid disodium salt | 0.2 | g/liter |
| Polyoxyethylene (13) naphthyl ether | 40.0 | g/liter |
| Potassium hydroxide | Amount giving pH 12.7 | |
| Water | Amount making 1 | liter |

(Gum solution (finisher solution) composition)

Plate protecting solution (Gum solution for 1 liter)

| | |
|---|---|
| White dextrin | 5.0% by weight |
| Hydroxypropyl starch | 10.0% by weight |
| Gum arabic | 1.0% by weight |
| Ammonium phosphate | 0.1% by weight |
| Sodium dilauryl succinate | 0.15% by weight |
| Polyoxyethylene naphthyl ether | 0.5% by weight |
| Polyoxyethylenepolyoxypropylene | 0.3% by weight |
| (50/50 by mol %) block copolymer | |
| (molecular weight 5000) | |
| Ethylene glycol | 1.0% by weight |
| Ethylenediaminetetraacetic acid | 0.005% by weight |
| disodium salt | |
| 1,2-Benzoisothiazoline-3-one | 0.005% by weight |

### (Image Formation)

The above-obtained light sensitive planographic printing plate material sample was imagewise exposed at a resolution of 2400 dpi (dpi represents the dot numbers per 2.54 cm) employing a plate setter installed with a 408 nm laser source with an output power of 30 mW, a CTP exposure apparatus (Tigercat produced by ECRM Co., Ltd.). An image pattern used for imagewise exposure had a solid image and a 50% square dot image with a screen line number of 175.

Subsequently, 300 m² of the exposed printing plate material sample was processed to give an area ratio of image portions to non-image portions of 1:9, employing a CTP automatic developing machine having a pre-heating section, a pre-washing section, a development section (with a developer tank charged with 22 liter of developer), an aqueous alkali solution processing section (with an aqueous alkali solution tank charged with 15 liter of aqueous alkali solution), a water washing section, a finisher-processing section, and a drying section in that order. During the processing, the developer was replenished with the developer replenisher in an amount of 50 ml per m² of the exposed printing plate material sample.

The light sensitive planographic printing plate material sample was processed employing a combination as shown in Table 1 of developer/developer replenisher described above and the compound of the invention represented by formula (1) to obtain planographic printing plate sample Nos. 1 through 7. Planographic printing plate samples obtained by processing the light sensitive planographic printing plate material sample with a developer and developer replenisher in which the compound represented by formula (1) was removed from the developer and developer replenisher as described above were regarded as comparative.

### (Evaluation of Contamination at Non-Image Portions and Dot Images)

Each of the resulting planographic printing plate sample Nos. 1 through 7 was mounted on a printing press DAIYA1F-1 (produced by Mitsubishi Jukogyo Co., Ltd.), and printing was carried out, where coated paper sheets, printing ink (soybean oil ink Naturalith 100 produced by Dainippon Ink Kagaku Kogyo Co., Ltd.), and dampening solution H solution SG-51 (concentration: 1.5%, produced by Tokyo Ink Co., Ltd.) were employed for printing. Contamination at non-image portions and ink receptivity at 97% dot image portions were visually observed, and evaluated as a measure of developability (or anti-contamination property). The results are shown in Table 1.

### Evaluation of Contaminations at Non-Image Portions

A: No contamination is observed.
B: Contamination at non-image portions is observed through a magnifying glass.
C: Contamination at non-image portions is visually observed.

### Quality of 97% Dot Image Portions

A: No clogging is observed at the image portions.
B: Clogging is observed at one or more of 100 dots of the image portions.
C: Clogging is observed at five or more of 100 dots of the image portions.

TABLE 1

| | | | Contam- | Quality | |
|---|---|---|---|---|---|
| | Developer/ | Compound | ination | of 97% | |
| | Developer | represented | at non- | dot | |
| Sample | replenisher | by | image | image | |
| No. | No. | formula (1) | portions | portions | Remarks |
| 1 | 1 | 1-1 | A | B | Inv. |
| 2 | 2 | 1-2 | A | A | Inv. |
| 3 | 3 | 1-3 | A | A | Inv. |
| 4 | 1 | 1-10 | A | A | Inv. |
| 5 | 2 | 1-12 | A | A | Inv. |
| 6 | 1 | None | B | C | Comp. |
| 7 | 2 | None | B | B | Comp. |

Inv.: Inventive,
Comp.: Comparative

As is apparent from Table 1, inventive samples 1 through 5, which are obtained by processing a light sensitive planographic printing plate material with a developer containing the compound of the invention, provide excellent dot image without contamination at non-image portions.

1. A developer for a light sensitive planographic printing plate material comprising an aluminum support and provided thereon, a light sensitive layer comprised of a light sensitive resin composition, the light sensitive planographic printing plate material after exposure being developed with the developer, wherein the developer contains a compound represented by the following formula (1), 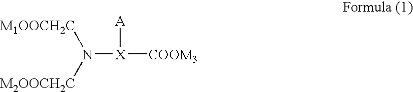
wherein A represents a substituted or unsubstituted alkyl group having a carbon atom number of from 1 to 5, a substituted or unsubstituted aryl group or COOM; M, M₁, M₂ and M₃ independently represent a hydrogen atom, an alkali metal or an ammonium group; and X represents CₘH₂ₘ₋₁ in which m represents an integer of from 1 to 5.

2. The developer for a light sensitive planographic printing plate material of claim 1, wherein A in formula (1) represents CH₃ or CH₂COOM₄ in which M₄ represents a hydrogen atom, an alkali metal or an ammonium group.

3. The developer for a light sensitive planographic printing plate material of claim 1, wherein A in formula (1) represents an alkyl group or an aryl group each having a hydroxyl group as a substituent.

4. The developer for a light sensitive planographic printing plate material of claim 1, wherein the developer contains alkali agents.

5. The developer for a light sensitive planographic printing plate material of claim 1, wherein the developer has a pH of from more than 8.5 to less than 13.0.

6. The developer for a light sensitive planographic printing plate material of claim 1, wherein the developer contains at least one compound selected from silic acid, phosphoric acid, carbonic acid, boric acid, phenols, saccharides, oximes, and fluorinated alcohols.

7. A method for processing a light sensitive planographic printing plate material, the method comprising the steps of developing a light sensitive planographic printing plate material with the developer of claim 1; and washing the developed planographic printing plate material with water.
